(19) United States
(12) Patent Application Publication
(10) Pub. No.: US 2026/0168902 A1
(43) Pub. Date: Jun. 18, 2026

(54) Soil Mass Coupling Test Apparatus and Test Method for Researching Full-Scale Strength of Soil Mass

(71) Applicant: SHIJIAZHUANG TIEDAO UNIVERSITY, Shijiazhuang (CN)

(72) Inventors: Chao YIN, Shijiazhuang (CN); Shuaiwei WANG, Shijiazhuang (CN); Cunbao ZHAO, Shijiazhuang (CN); Wei YUAN, Shijiazhuang (CN); Wei WANG, Shijiazhuang (CN)

(21) Appl. No.: 19/531,424

(22) Filed: Feb. 5, 2026

Related U.S. Application Data
(63) Continuation of application No. PCT/CN2024/144391, filed on Dec. 31, 2024.

(30) Foreign Application Priority Data
Dec. 2, 2024 (CN) 202411749179.0

(51) Int. Cl. G01N 3/24; G01N 3/06; G01N 33/24

(52) U.S. Cl. CPC G01N 3/24; G01N 3/068; G01N 33/24; G01N 2203/0222; G01N 2203/0242

(57) ABSTRACT

The present application provides a soil mass coupling test apparatus and a test method for researching full-scale strength of soil mass. The soil mass coupling test apparatus includes a soil mass testing machine, a linear rail, a box body, and an environment simulation assembly. The soil mass testing machine includes a testing platform and a loading assembly, which is configured to conduct one or more tests selected from tension test, compression-shear test, and tension-shear test on the soil mass samples, thereby testing the properties of the soil mass. The box body is movably installed on the linear rail and features a testing chamber for accommodating the soil mass sample and at least partially accommodating the loading assembly. The environmental simulation assembly adjusts the testing chamber to the appropriate temperature and humidity.

### CROSS-REFERENCE TO RELATED APPLICATIONS

This application is a continuation of International Application No. PCT/CN2024/144391, filed on Dec. 31, 2024, entitled “Soil Mass Coupling Test Apparatus and Test Method for Researching Full-Scale Strength of Soil Mass”, which claims priority to Chinese Patent Application No. 202411749179.0, filed on Dec. 2, 2024, entitled “Soil Mass Coupling Test Apparatus and Test Method for Researching Full-Scale Strength of Soil Mass”. The disclosures of the aforementioned applications are hereby incorporated by reference in their entireties.

### TECHNICAL FIELD

The present application relates to the technical field of soil mass coupling test, and particularly relates to a soil mass coupling test apparatus and a test method for researching full-scale strength of soil mass.

### BACKGROUND

Failure of engineering soil mass is not only related to factors such as changes of groundwater levels, increase of external loads, earthquakes, but also intimately linked to the ambient environmental regime. For instance, in high temperature weather, high air temperature and low relative humidity accelerate water evaporation and reduce water content in the soil mass. In cold wave weather, water in the soil mass freezes and expands. Under foggy or prolonged rainy conditions, the relative humidity remains high. As the temperature drops, water vapor in air condenses into droplets that infiltrate the soil mass, raising its moisture content. Because these environmental variations continuously alter the state of the soil mass, its mechanical properties changes accordingly.

In prior art, tension and shear coupling tests on soil mass are conducted exclusively in a laboratory, where the ambient conditions differ markedly from those in the field. Consequently, the results of such laboratory-based coupling tests inevitably deviate from real-world behavior. Therefore, investigating how varying environmental conditions influence soil mechanical properties is essential for guiding engineering practice and improving project quality.

### SUMMARY

These and other problems are generally solved or circumvented, and technical advantages are generally achieved, by embodiments of the present application which provide a soil mass coupling test apparatus and a test method for researching full-scale strength of soil mass.

### Technical Problems

The present application provides a soil mass coupling test apparatus and a test method for researching full-scale strength of soil mass to solve the problem that the coupling tests of the soil mass are all conducted in a laboratory, resulting in deviations between test results and actual conditions in the prior art.

### Technical Solutions

The present application adopts the following technical solutions:

In a first aspect, the present application provides a soil mass coupling test apparatus including a soil mass testing machine, a linear rail, a box body, and an environment simulation assembly; the soil mass testing machine includes a testing platform and a loading assembly arranged on the testing platform; the linear rail is arranged horizontally at one side of the soil mass testing machine; the box body is slidably arranged on the linear rail, where the box body includes a testing chamber which has an inlet-outlet on a side facing towards the soil mass testing machine, and the inlet-outlet is movably connected with a box door of the box body; the box body has a testing position and an idle position, and the loading assembly is at least partially accommodated within the testing chamber when the box body is in the testing position, and the loading assembly is disengaged from the testing chamber when the box body is in the idle position; the environment simulation assembly includes a temperature control module and a humidity control module disposed in the box body; the temperature control module is configured to adjust a temperature of the testing chamber, and the humidity control module is configured to adjust a humidity of the testing chamber.

In one of the embodiments, a wall of the testing chamber is provided with a heat exchange hole connected with an air duct; an output end of the temperature control module and an output end of the humidity control module is respectively connected with the air duct.

In one of the embodiments, the box body further includes an equipment accommodating chamber; the temperature control module and the humidity control module are arranged in the equipment accommodating chamber; and the equipment accommodating chamber is in communication with the testing chamber through the air duct and the heat exchange hole.

In one of the embodiments, the soil mass coupling test apparatus further includes a multi-blade centrifugal fan and a frequency converter arranged in the air duct to realize a dynamic regulation of an air volume; and a flow deflector arranged in the air duct or the testing chamber; where a cross-sectional shape of the air duct is circular, the air duct is configured to supply air from a top of the testing chamber and returns air from a bottom of the testing chamber to form an vertical air flow, and the temperature control module and the humidity control module are respectively in communication with the air duct through a temperature channel and a humidity channel.

In one of the embodiments, the environmental simulation assembly further includes an instrument detection module; and the instrument detection module is connected with the temperature control module and the humidity control module, and is configured to detect components in the temperature control module and the humidity control module.

In one of the embodiments, the soil mass testing machine further includes a reaction frame, and the reaction frame is arranged on the testing platform and configured to form a testing area together with an upper surface of the testing platform; the testing area includes a tension-shear zone, a tension zone and a compression-shear zone; the loading assembly includes a first shear testing assembly arranged in the tension-shear zone, a tension testing assembly arranged in the tension zone, and a second shear testing assembly arranged in the compression-shear zone.

In one of the embodiments, the reaction frame includes a top plate and two side plates; the top plate is disposed above the testing platform; the two side plates are arranged opposite to each other and connected between the top plate and the testing platform; the top plate, the two side plates and the upper surface of the testing platform enclose to form the testing area; the tension-shear zone is arranged adjacent to a first side plate of the two side plates, and the compression-shear zone is arranged adjacent to a second side plate of the two side plates.

In one of the embodiments, the first shear testing assembly includes a first slide rail, a first soil mass specimen former, a first shear loading unit and a first shear top block; the first slide rail is arranged horizontally on the testing platform and located in the tension-shear zone, and the first soil mass specimen former is slidably arranged on the first slide rail; the first shear loading unit is arranged on the reaction frame and has a first extensible end extendable and retractable along a length direction of the first slide rail, and the first extensible end is provided with a first shear block; the first shear top block is arranged on the testing platform, where the first shear top block and the first shear block are arranged opposite to each other on two sides of the first soil mass specimen former and staggered in an vertical direction.

In one of the embodiments, the first shear testing assembly, the tension testing assembly, and the second shear testing assembly are arranged linearly; the reaction frame has an adjustment slot above the first shear test assembly, the tension testing assembly and the second shear testing assembly; the loading assembly further includes a tension-compression loading assembly slidably arranged in the adjustment slot, and the tension-compression loading assembly has a lifting end vertically movable.

In one of the embodiments, the first soil mass specimen former includes a first connecting block, a first confining cylinder, a second confining cylinder and a second connecting block; the first connecting block is slidably fitted to the first slide rail, and the first confining cylinder is disposed above the first connecting block and detachably connected with the first connecting block; the second confining cylinder is disposed above the first confining cylinder and detachably connected with the first confining cylinder; the second connecting block is disposed above the second confining cylinder and detachably connected with the second confining cylinder; an upper part of the second connecting block is detachably connected to the lifting end; the first connecting block, the first confining cylinder, the second confining cylinder and the second connecting block form a molding chamber for a soil mass sample, and a radial cross-section of the molding chamber is circular, and a diameter of the radial cross-section of the molding chamber gradually decreases from two ends towards a middle of the molding chamber.

In one of the embodiments, the soil mass coupling test apparatus further includes a temperature monitoring element and a humidity monitoring element disposed on a side of the first connecting block that forms the molding chamber, or a temperature monitoring element and a humidity monitoring element disposed on a side of the second connecting block that forms the molding chamber; the temperature monitoring element disposed on the side of the first connecting block or on the side of the second connecting block is configured to monitor a temperature of the soil mass sample, and the humidity monitoring element disposed on the side of the first connecting block or on the side of the second connecting block is configured to monitor a humidity of the soil mass sample.

In one of the embodiments, the first connecting block and the first constraining cylinder are connected together by a first snap-fit structure, and the second connecting block and the second constraining cylinder are connected together by a second snap-fit structure.

In one of the embodiments, the first constraining cylinder and the second constraining cylinder are respectively formed by a plurality of arc-shaped plates enclosed and fixed together by at least one hoop ring.

In one of the embodiments, the tension-compression loading assembly is provided with a laser level, and a laser direction of the laser level is vertical.

In one of the embodiments, the box body is provided with an avoidance opening configured for avoiding the loading assembly, and the box body is further provided with a blocking element configured for opening or closing the avoidance opening.

In one of the embodiments, the soil mass coupling test apparatus further includes a sealing ring detachably arranged, where when the loading assembly is extended into the testing chamber through the avoidance opening, the sealing ring is arranged around the loading assembly from an outside of the box body to seal the avoidance opening.

In one of the embodiments, the soil mass coupling test apparatus further includes a stopper, and the stopper is configured for locking the box body at a position of the linear rail.

In one of the embodiments, the stopper includes: a mounting base arranged on an outer wall of the box body, and a first claw and a second claw rotatably arranged on the mounting base; the first claw and the second claw are configured for forming a clamping space for clamping the linear rail.

In a second aspect, the present application further provides a test method for researching full-scale strength of soil mass, which adopts the soil mass coupling test apparatus according to any one of the aforesaid embodiments, including the following steps:
determining a temperature and a humidity required for testing;
fixing a prepared soil mass sample on the loading assembly, and moving the box body from the idle position to the testing position, to accommodate the prepared soil mass samples in the testing chamber of the box body;
closing the box door of the box body, setting temperature parameters of the temperature control module and humidity parameters of the humidity control module based on a temperature and a humidity required for testing;
waiting until a temperature and a humidity of the prepared soil mass sample tend to be stable, controlling the loading assembly to perform a soil mass coupling test on the prepared soil mass sample, and saving images and data of a test result; and
after the soil mass coupling test is completed, adjusting the temperature control module to a cooling state to freeze the prepared soil mass sample that has been tested, and preserving a failure state of the prepared soil mass sample.

In one of the embodiments, a plurality of prepared soil mass samples are provided, where the loading assembly is configured to perform a tension test, a tension-shear test, and a compression-shear test on the plurality of prepared soil mass samples respectively, and after soil mass coupling tests on the plurality of prepared soil mass samples are completed, a strength envelope of the plurality of prepared soil mass samples is plotted based on test results.

### Advantageous Effects of the Disclosure

Compared with the prior art, the advantageous effects of the soil mass coupling test apparatus provided in the present application are as follows:

The soil mass coupling test apparatus provided in the present application includes a soil mass testing machine, a linear rail, a box body, and an environment simulation assembly. The soil mass testing machine includes a testing platform and a loading assembly which is used to conduct one or more tests selected from tension test, compression-shear test, and tension-shear test on the soil mass samples, thereby testing the properties of the soil mass. The box body is movably installed on the linear rail and features a testing chamber for accommodating the soil mass sample and at least partially accommodating the loading assembly. The environmental simulation assembly adjusts the testing chamber to the appropriate temperature and humidity, enabling the simulation of the physical properties and mechanical behavior of the soil mass samples under various environments such as high temperatures, heavy fog, and rainy days, in order to better guide engineering practice.

In the present application, the box body adopts a movable design, which is moved to the testing position during the test. Before testing, the soil mass sample may be placed inside the testing chamber first, allowing the soil mass sample to gradually reach the required temperature and humidity. Then, the box body is moved to the testing position, and the soil mass sample is installed on the loading assembly for testing. This arrangement helps shorten the time the loading assembly remains in the testing chamber, thereby preventing the components of the loading assembly from being damaged due to the effects of the temperature or humidity of the testing chamber, and extending the service life of the loading assembly.

In the present application, the temperature control module is capable of regulating the temperature in the testing chamber. After the test is completed, the temperature control module can cool the testing chamber to freeze the soil mass sample, preserving its failure state. The mesoscopic deformation and microstructure of the soil mass can be further investigated by Computed Tomography (CT) scanning, Scanning Electron Microscope (SEM) imaging, X-ray Diffraction (XRD) analysis, and transmission electron microscopy, facilitating further research on the soil mass.

The test method for researching full-scale strength of soil mass is implemented using the soil mass coupling test apparatus described in any of the embodiments, and it achieves the same technical effects as those described. Therefore, no further elaboration is given here.

### BRIEF DESCRIPTION OF THE DRAWINGS

FIG. 1 is a structural schematic diagram of a soil mass coupling test apparatus provided in one embodiment of the present application;

FIG. 2 is an enlarged view of section A in FIG. 1;

FIG. 3 is a structural schematic diagram of a soil mass testing machine provided in another embodiment of the present application;

FIG. 4 is a structural schematic diagram of a first soil mass specimen former provided in another embodiment of the present application;

FIG. 5 is a structural schematic diagram of a second soil mass specimen former provided in another embodiment of the present application;

FIG. 6 shows a strength envelope of soil mass drawn using the test method for researching full-scale strength of soil mass provided by the present application;

FIG. 7 is a schematic layout diagram of a testing chamber and an equipment accommodating chamber according to one embodiment of the present application;

FIG. 8 is a structural schematic diagram of a first confining cylinder according to one embodiment of the present application;

FIG. 9 is a structural schematic diagram of a tension-compression loading assembly provided with a laser level according to one embodiment of the present application;

FIG. 10 is a structural schematic diagram of a loading assembly passing through a side wall of a box body according to one embodiment of the present application; and

FIG. 11 is a structural schematic diagram of a stopper according to one embodiment of the present application.

In the drawings:

1, soil mass testing machine; 2, linear rail; 3, box body; 31, testing chamber; 311, inlet-outlet; 312, heat exchange hole; 32, equipment accommodating chamber; 33, air duct; 34, flow deflector; 35, temperature channel; 36, humidity channel; 37, avoidance opening; 4, box door; 5, environment simulation assembly; 51, temperature control module; 52, humidity control module; 53, instrument detection module; 6, multi-blade centrifugal fan; 7, frequency converter; 8, sealing ring; 9, stopper; 91, mounting base; 92, first claw; 93, second claw; 10, testing platform; 20, loading assembly; 21, first shear testing assembly; 211, first slide rail; 212, first soil mass specimen former; 2121, first connecting block; 2122, first confining cylinder; 2123, second confining cylinder; 2124, second connecting block; 2125, temperature monitoring element; 2126, humidity monitoring element; 2127, arc-shaped plate; 2128, hoop ring; 213, first shear loading unit; 214, first shear top block; 22, tension testing assembly; 23, second shear testing assembly; 231, second slide rail; 232, second soil mass specimen former; 2321, third connecting block; 2322, fourth connecting block; 2323, third confining cylinder; 2324, fourth confining cylinder; 2325, fifth confining cylinder; 233, second shear loading unit; 234, second shear top block; 24, tension-compression loading assembly; 241, laser level; 30, reaction frame; 31, top plate; 32, side plate; 40, soil mass sample.

### DETAILED DESCRIPTION OF ILLUSTRATIVE EMBODIMENTS

In the following description, a clear and complete description of the technical solution in the embodiments of the present application will be provided referring to the drawings. It should be appreciated, however, that the concepts disclosed herein can be embodied in a wide variety of specific contexts. The specific embodiments discussed are merely illustrative, and do not limit the scope of the claims.

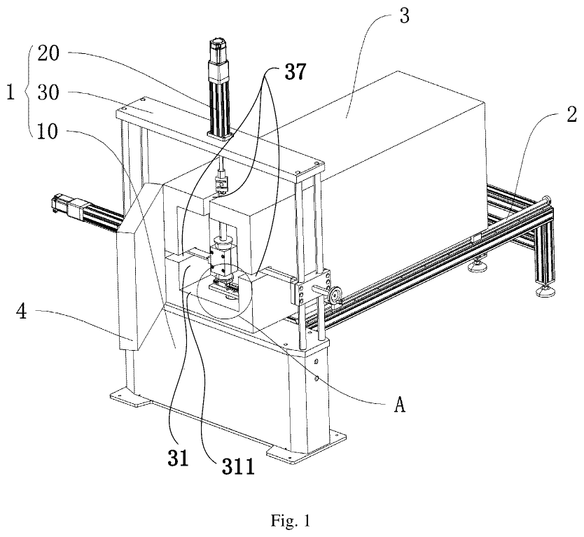

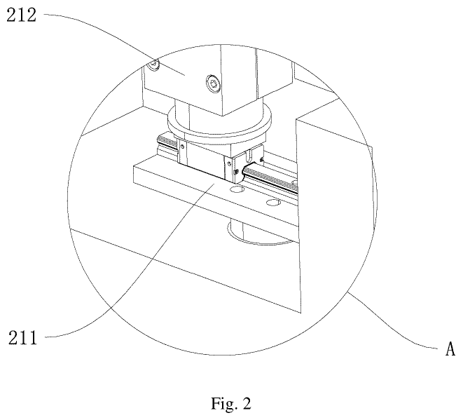

Referring to FIGS. 1 to 11, the following provides a description of the soil mass coupling test apparatus provided in the embodiments of the preset application, as well as the test method for researching full-scale strength of soil mass.

As shown in FIGS. 1 to 7, in the first aspect, the embodiment of the present application provides a soil mass coupling test apparatus, which includes a soil mass testing machine 1, a linear rail 2, a box body 3, and an environment simulation assembly 5. The soil mass testing machine 1 includes a testing platform 10 and a loading assembly 20 arranged on the testing platform 10. The linear rail 2 is arranged horizontally at one side of the soil mass testing machine 1. The box body 3 is slidably arranged on the linear rail 2, where the box body 3 includes a testing chamber 31 which has an inlet-outlet 311 on the side facing towards the soil mass testing machine 1, and the inlet-outlet 311 is movably connected with a box door 4 of the box body 3; the box body 3 has a testing position and an idle position, and the loading assembly 20 is at least partially accommodated within the testing chamber 31 when the box body 3 is in the testing position, and the loading assembly 20 is disengaged from the testing chamber 31 when the box body 3 is in the idle position. The environment simulation assembly 5 includes a temperature control module 51 and a humidity control module 52 disposed in the box body 3. The temperature control module 51 is configured to adjust the temperature of the testing chamber 31, and the humidity control module 52 is configured to adjust the humidity of the testing chamber 31.

Compared with the prior art, the advantageous effects of the soil mass coupling test apparatus provided in the embodiment of the present application are as follows:

The soil mass coupling test apparatus provided in the embodiment of the present application includes a soil mass testing machine 1, a linear rail 2, a box body 3, and an environment simulation assembly 5. The soil mass testing machine 1 includes a testing platform 10 and a loading assembly 20 which is used to conduct one or more tests selected from tension test, compression-shear test, and tension-shear test on the soil mass samples 40, thereby testing the properties of the soil mass. The box body 3 is movably installed on the linear rail 2 and features a testing chamber 31 for accommodating the soil mass sample 40 and at least partially accommodating the loading assembly 20. The environmental simulation assembly 5 adjusts the testing chamber 31 to the appropriate temperature and humidity, enabling the simulation of the physical properties and mechanical behavior of the soil mass samples 40 under various environments such as high temperatures, heavy fog, and rainy days, in order to better guide engineering practice and improve engineering quality.

In the embodiment of this application, the box body 3 adopts a movable design, which is moved to the testing position during the test. Before testing, the soil mass sample 40 may be placed inside the testing chamber 31 first, allowing the soil mass sample 40 to gradually reach the required temperature and humidity. Then, the box body 3 is moved to the testing position, and the soil mass sample 40 is installed on the loading assembly 20 for testing. This arrangement helps shorten the time the loading assembly 20 remains in the testing chamber 31, thereby preventing the components (such as force sensors, fixtures, etc.) of the loading assembly 20 from being damaged due to the effects of the temperature or humidity of the testing chamber 31, and extending the service life of the loading assembly 20.

In the embodiment of the present application, the temperature control module 51 is capable of regulating the temperature in the testing chamber 31. After the test is completed, the temperature control module 51 can cool the testing chamber 31 to freeze the soil mass sample 40, preserving its failure state. The mesoscopic deformation and microstructure of the soil mass can be further investigated by Computed Tomography (CT) scanning, Scanning Electron Microscope (SEM) imaging, X-ray Diffraction (XRD) analysis, and transmission electron microscopy, facilitating further research on the soil mass.

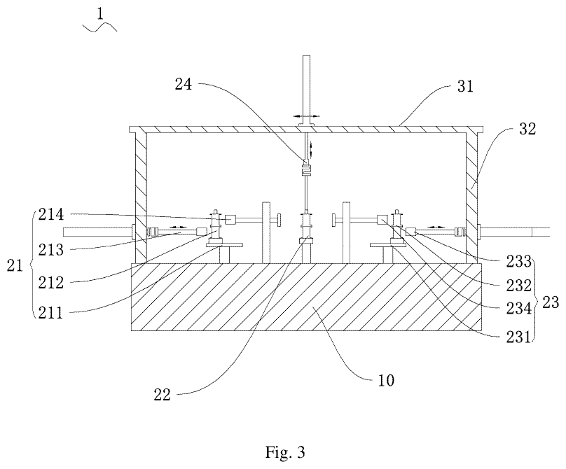

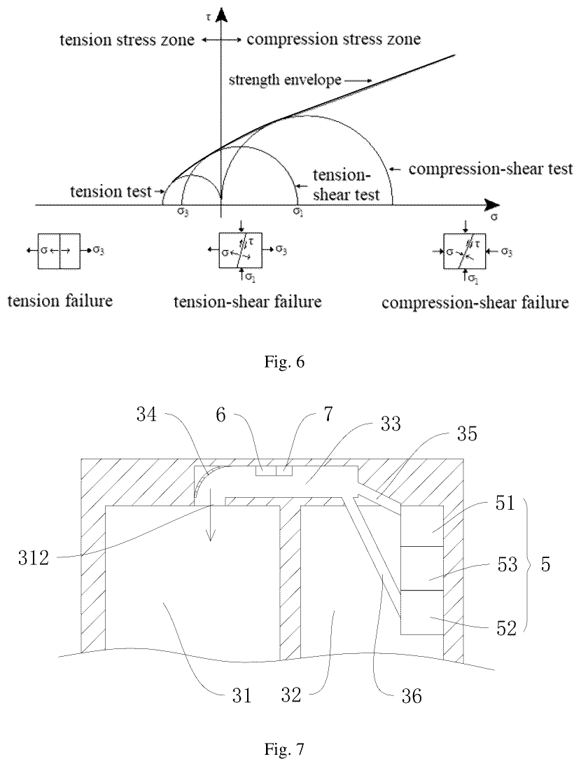

The soil mass testing machine 1 is a multifunctional testing device for soil mass. According to different testing requirements, the soil mass testing machines 1 with various models and specifications may be selected for conducting tests such as tension tests, tension-shear tests, and compression-shear tests on soil mass samples 40. The soil mass testing machine 1 includes a testing platform 10 and a loading assembly 20 arranged on the testing platform 10. The loading assembly 20 may include a tension loading assembly, a compression-shear loading assembly, and a tension-shear loading assembly. The loading assembly 20 may include three types of the loading assembly, or only one or two types of the loading assembly, allowing for flexible loading configurations through component replacement or selective usage, and there are no specific restrictions. For example, the loading assembly 20 shown in FIG. 1 can conduct tension tests, tension-shear tests, and compression-shear tests. However, it should be noted that due to the presence of single soil mass specimen former, only a single test can be conducted at a time. Another example is the loading assembly 20 shown in FIG. 3, which can also conduct tension tests, tension-shear tests, and compression-shear tests. However, it should be noted that due to the presence of three soil mass specimen formers, tests can be conducted one by one sequentially. The soil mass coupling test apparatus provided in the present application can realize the test of soil sample 40 under the full stress states of compression-shear, tension-shear and uniaxial tension. The same type soil sample 40 can be subjected to compression-shear, tension-shear and uniaxial tension tests without changing its size, diameter, moisture content and other parameters. The full-stress strength envelope of soil sample 40 can be obtained through these three groups of tests, as shown in FIG. 6.

The linear rail 2 is arranged horizontally at one side of the testing platform 10. Specifically, the linear rail 2 may be circular, square, or other shape, as long as it can facilitate the smooth movement of the box body 3. The box body 3 is slidably arranged on the linear rail 2 driven manually or by driving components such as hydraulic push rods. The box body 3 has a testing chamber for accommodating the soil mass sample 40 and the loading assembly 20. When the loading assembly 20 is only partially accommodated in the testing chamber 31 as shown in FIG. 1, the wall of the testing chamber 31 should be provided with a clearance hole or a clearance slot allowing the corresponding parts of the loading assembly 20 to pass through.

The box door 4 is movably positioned at the inlet-outlet 311. Specifically, the box door 4 may be connected to the box body 3 using methods such as screw connection, plug connection, or hinged rotation connection. It should be understood that the box door 4 is designed in such a way that it will not interfere with the loading assembly 20 entering the box body 3. To facilitate observation of the interior, an observation window with a transparent glass plate may be installed on the box door 4.

It is understandable that before conducting the test, the soil mass sample 40 is usually placed in the testing chamber 31 for a period of time to achieve the required temperature, humidity, and other parameters. Alternatively, the soil mass sample 40 can be installed on the loading assembly 20 first, and then the box body 3 is moved to the testing position. In this way, both the soil mass sample 40 and the loading assembly 20 are in the temperature and humidity environment simulated by the testing chamber 31. Alternatively, the soil mass sample 40 can be placed in the testing chamber 31 first, and after the temperature and humidity of the soil mass sample 40 reach the requirements, the box body 3 is moved to the testing position. The soil mass sample 40 is installed on the loading assembly 20 for a coupling test. This approach reduces the time the loading assembly 20 stays in the testing chamber 31, and the related components are less susceptible to factors such as temperature and humidity.

The environment simulation assembly 5 includes a temperature control module 51 and a humidity control module 52. The temperature control module 51 specifically includes a refrigeration unit and a heating unit, while the humidity control module 52 includes a humidifier. The refrigeration unit may employ compressor for cooling, while the heating unit can be a heat pump or an electric heater. A circulation fan and inlet/outlet holes are installed inside the testing chamber 31 to achieve airflow, ensuring uniform temperature and humidity throughout the testing chamber 31. The humidifier is equipped with a water tank and a water-level indicator. Operators should promptly refill the water tank whenever it runs low.

The environment simulation assembly 5 can be controlled through a liquid crystal touch display screen, with a humidity adjustment range from 15% to 95% relative humidity (RH) and a temperature adjustment range from −30° C. to 60° C.

Referring to FIGS. 1 and 7, in some possible embodiments, the box body 3 further includes an equipment accommodating chamber 32. The temperature control module 51 and the humidity control module 52 are respectively arranged inside the equipment accommodating chamber 31. The wall of the testing chamber 31 is provided with a heat exchange hole 312 connected with an air duct 33. The air inlet end of the air duct 33 is arranged inside the equipment accommodating chamber 32. The output end of the temperature control module 51 and the output end of the humidity control module 52 are respectively connected with the air duct 33. A circulating fan is arranged inside the air duct 33. After being mixed evenly in the air duct 33, the airflow enters the testing chamber 31 through the heat exchange hole 312.

For example, as shown in FIG. 7, the soil mass coupling test apparatus further includes a multi-blade centrifugal fan 6 and a frequency converter 7 arranged in the air duct 33 to realize a dynamic regulation of an air volume, control the temperature within ±0.5° C., and achieve precise regulation of temperature and humidity. The cross-sectional shape of the air duct 33 is circular; and the air duct 33 is configured to supply air from the top of the test chamber 31 and return air from the bottom of the test chamber 31 to form vertical air flow, thereby generating an orderly unidirectional flow. This design not only improves the uniformity of air supply but also avoids local circulation caused by disordered flow directions. The soil mass coupling test apparatus further includes a flow deflector 34 arranged in the air duct 33 and/or the test chamber 31, which can guide the air flow to move along a preset path and prevent the formation of eddy currents. The temperature control module 51 and the humidity control module 52 are respectively in communication with the air duct 33 through a temperature channel 35 and a humidity channel 36; the temperature channel 35 and the humidity channel 36 are separated from each other, enabling independent control of temperature and humidity variations. Through the aforesaid design, the environmental simulation assembly 5 can achieve precise control and locking of temperature and humidity, thereby ensuring the stability of the internal environment of the test chamber 31.

For example, the environmental simulation assembly 5 may further include an instrument detection module 53, which is connected with the temperature control module 51 and the humidity control module 52 and is configured to detect the components in the temperature control module 51 and the humidity control module 52. The instrument detection module 53 can timely identify component faults, aging or abnormal working states, avoiding the degradation of environmental simulation accuracy or system shutdown caused by the failure of temperature and humidity control components. The instrument detection module 53 may include current/voltage sensors, temperature sensors, humidity sensors, rotation speed sensors, single-chip microcomputers, signal amplifiers, signal converters, alarms, indicator lights, communicators, etc.

Referring to FIGS. 1 and 3, in some possible embodiments, the soil mass testing machine 1 further includes a reaction frame 30, and the reaction frame 30 is arranged on the testing platform 10 and configured to form a testing area together with an upper surface of the testing platform 10, the testing area including a tension-shear zone, a tension zone and a compression-shear zone; the loading assembly 20 includes a first shear testing assembly 21 arranged in the tension-shear zone, a tension testing assembly 22 arranged in the tension zone, and a second shear testing assembly 23 arranged in the compression-shear zone. As shown in FIG. 3, the first shear testing assembly 21, the tension testing assembly 22, and the second shear testing assembly 23 may either share a single translatable tension-compression loading assembly 24, or each be equipped with a separate tension-compression loading assembly 24. There are no restrictions on this configuration.

In this embodiment, the testing area is functionally divided into a tension-shear zone, a tension zone, and a compression-shear zone, which are respectively used to perform tension-shear tests, tension tests, and compression-shear tests on soil mass samples 40, thereby allowing for mechanical property experiments on soil mass samples 40, including uniaxial tension, tension-shear, and compression-shear. During uniaxial tension and tension-shear tests, the shape of the soil mass samples 40 is designed to be thick at both ends and thin in the middle, with a circular radial cross-section and a cylindrical middle section. The overall height of the soil mass sample 40 is 120 millimeters (mm), the height of the cylindrical middle section is 20 mm and its diameter is 39 mm. Since no tension force is applied during the compression-shear tests, the soil mass sample 40 can be designed to have the same dimensions as the aforementioned cylindrical middle section, i.e., 20 mm in height and 39 mm in diameter.

The tension tests, tension-shear tests, and compression-shear tests can be conducted either simultaneously or sequentially. Since the middle section of the soil mass samples 40 used in these three tests have the same shape and size, the loading assembly 20 can achieve experimental measurement of the complete strength envelope of the soil mass as shown in FIG. 6, thereby providing a theoretical basis for engineering practice. The loading assembly 20 can be driven by a servo motor controlled by a controller, which offers high control accuracy, good stability, and saves time and labor. It is also equipped with data recording and data processing software, enabling real-time data recording and processing.

It should be noted that during tension and tension-shear tests, the soil mass sample 40 is designed to be thick at both ends and thin in the middle. This design allows both ends of the soil mass sample 40 to be partially seated in the upper and lower clamps, respectively, so that, it allows for a true simulation of the tension state of the soil mass sample 40 during tension test, thereby ensuring the accuracy of the test results.

Referring to FIGS. 1 and 3, in some possible embodiments, the reaction frame 30 includes a top plate 31 and two side plates 32. The top plate 31 is disposed above the testing platform 10. The two side plates 32 are arranged opposite to each other and connected between the top plate 31 and the testing platform 10; the top plate 31, the two side plates 32, and the upper surface of the testing platform 10 enclose to form the testing area; the tension-shear zone is arranged adjacent to a first side plate of the two side plates 32, and the compression-shear zone is arranged adjacent to a second side plate of the two side plates 32. The tension zone is set between the tension-shear zone and the compression-shear zone.

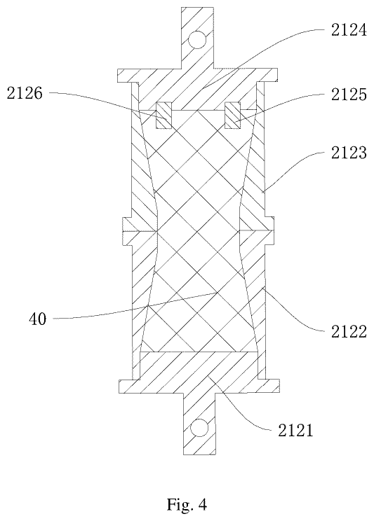

Referring to FIGS. 3 and 4, in some possible embodiments, the first shear testing assembly 21 includes a first slide rail 211, a first soil mass specimen former 212, a first shear loading unit 213, and a first shear top block 214. The first slide rail 211 is arranged horizontally on the testing platform 10 and located in the tension-shear zone, and the first soil mass specimen former 212 is configured to prepare soil mass sample 40 and is slidably arranged on the first slide rail 211. The first shear loading unit 213 is arranged on the reaction frame 30 and has a first extensible end extendable and retractable along a length direction of the first slide rail 211, and the first extensible end is provided with a first shear block. The first shear top block 214 is arranged on the testing platform 10, where the first shear top block 214 and the first shear block are arranged opposite to each other on two sides of the first soil mass specimen former 212 and staggered in the vertical direction.

In this embodiment, the first shear testing assembly 21 includes a first slide rail 211, a first soil mass specimen former 212, a first shear loading unit 213, and a first shear top block 214. The first shear loading unit 213 and the first shear top block 214 are arranged opposite to each other relative to the first soil mass specimen former 212, and are capable of moving along their respective length directions, thereby applying a shear force to the soil mass sample 40. The movement of the first shear loading unit 213 and the first shear top block 214 may be automatically controlled using power devices such as servo motors and hydraulic cylinders, or manually adjusted using structures such as lead screws and handwheels. A pressure sensor is installed between the first extensible end and the first shear block to measure the pressure applied by the first shear block to the soil mass sample 40.

The first soil mass specimen former 212 is used for preparing soil mass samples 40 of specific shapes. The lower end of the first soil mass specimen former 212 is slidably mounted on the first slide rail 211, and the upper end of the first soil mass specimen former 212 is connected with the tension-compression loading assembly 24. The tension-compression loading assembly 24 may be driven by servo motors and hydraulic cylinders, and includes a displacement sensor and a tension sensor, which is used to apply a tension force or compression force to the soil mass sample 40 in the vertical direction.

It should be noted that the first soil mass specimen former 212 has a forming mode and a testing mode. In the forming mode, the first soil mass specimen former 212 itself can form a closed molding chamber, used to form a soil mass sample 40 of a specific shape. When a test is required, the arc-shaped plate or sleeve, etc., that encloses and forms the molding chamber can, as needed, be removed from the first soil mass specimen former 212, switching the first soil mass specimen former 212 in the testing mode. During the test, a shear ring is added around the outer circumference of the soil mass sample 40, and the first shear loading unit 213 applies shear force to the soil mass sample 40 through the shear ring. Of course, when the arc-shaped plate does not affect the normal test, it may be left in place, and the operator can decide based on the actual situation.

Referring to FIG. 3, in some possible embodiments, the first shear testing assembly 21, the tension testing assembly 22, and the second shear testing assembly 23 are arranged linearly. The reaction frame 30 has an adjustment slot above the first shear testing assembly 21, the tension testing assembly 22, and the second shear testing assembly 23. The loading assembly 20 further includes a tension-compression loading assembly 24 slidably arranged in the adjustment slot, and the tension-compression loading assembly 24 has a lifting end vertically movable. In this embodiment, the first shear testing assembly 21, the tension testing assembly 22, and the second shear testing assembly 23 may share a single tension-compression loading assembly 24.

In this embodiment, the tension-compression loading assembly 24 can move along the adjustment slot among the tension-shear zone, the tension zone, and the compression-shear zone, enabling tension-shear tests, tension tests, or compression-shear tests to be conducted, respectively. This design not only simplifies the device structure and saves manufacturing costs, but also eliminates test errors caused by using different instruments, thereby improving test precision and the accuracy of results.

Referring to FIGS. 3 and 4, in some possible embodiments, the first soil mass specimen former 212 includes a first connecting block 2121, a first confining cylinder 2122, a second confining cylinder 2123, and a second connecting block 2124. The first connecting block 2121 is slidably fitted to the first slide rail 211, and the first confining cylinder 2122 is disposed above the first connecting block 2121 and detachably connected with the first connecting block 2121. The second confining cylinder 2123 is disposed above the first confining cylinder 2122 and detachably connected with the first confining cylinder 2122. The second connecting block 2124 is disposed above the second confining cylinder 2123 and detachably connected with the second confining cylinder 2123; an upper part of the second connecting block 2124 is detachably connected with the lifting end of the tension-compression loading assembly 24. The first connecting block 2121, the first confining cylinder 2122, the second confining cylinder 2123, and the second connecting block 2124 form a molding chamber for the soil mass sample 40, and a radial cross-section of the molding chamber is circular, and a diameter of the radial cross-section of the molding chamber gradually decreases from two ends towards the middle of the molding chamber.

In this embodiment, the first soil mass specimen former 212 can also be used to prepare soil mass samples 40 for tension tests. The tension-compression loading assembly 24 is connected to the top end of the first soil mass specimen former 212 (i.e., the second connecting block 2124) and is capable of applying a certain tension force to conduct tension tests or tension-shear tests. It is understandable that when the first slide rail 211 and the first shear loading unit 213 in the first shear testing assembly 21 are inactive, a tension test can still be conducted using the first soil mass specimen former 212 and the tension-compression loading assembly 24. Of course, considering the potential instability caused by the first slide rail 211, the tension test is usually conducted using the tension testing assembly 22 located in the middle position. The tension testing assembly 22 includes a soil mass specimen former identical in construction to the first soil mass specimen former 212 but fixed in position. Of course, when the tension-compression loading assembly 24 is inactive, a shear test can be conducted using the first slide rail 211, the first shear loading unit 213, and the first shear top block 214.

In this embodiment, the first connecting block 2121 and the first confining cylinder 2122, as well as the second confining cylinder 2123 and the second connecting block 2124, may be connected by threaded connections. The first confining cylinder 2122 and the second confining cylinder 2123 are arranged in mutual contact and are detachably connected at their mating surface with screws. During a test, the screws between the first confining cylinder 2122 and the second confining cylinder 2123 are removed. The first confining cylinder 2122 and the second confining cylinder 2123 can be integrated or an assembly formed by enclosing multiple arc-shaped plates, with detachable connections between adjacent arc-shaped plates achieved through clamping, screw connection, or the like. When the soil mass sample 40 is prepared and installed on the loading assembly 20, the first confining cylinder 2122 and the second confining cylinder 2123 can be taken off, and then a shear ring can be installed to the outer circumference of the soil mass sample 40. Of course, the first confining cylinder 2122 and the second confining cylinder 2123 may also be left in place if they do not affect normal testing.

As shown in FIG. 4, the first confining cylinder 2122 and the second confining cylinder 2123 have the same structure. The following gives a detailed description of the first confining cylinder 2122. Specifically, the first confining cylinder 2122 includes a first molding cavity and a second molding cavity, which are connected with each other and penetrate the first confining cylinder 2122 along its axial direction. The first molding cavity is conical. The first end of the first molding cavity is connected with the first connecting block 2121 and the second end of the first molding cavity is connected with the second molding cavity. The second molding cavity is cylindrical with an inner diameter of 39 mm and a height of 10 mm.

During the tension-shear test, the soil mass sample 40 is fixed on the lower first slide rail 211, which can effectively reduce friction and make the first shear block and the first shear top block 214 closely adhere to the soil mass sample 40. The first connecting block 2121 is arranged on the slide block, which is in sliding fit with the first slide rail 211, allowing the slide block and the soil mass sample 40 to move synchronously under shear.

Referring to FIG. 4, in some possible embodiments, a temperature monitoring element 2125 and a humidity monitoring element 2126 are disposed on a side of the first connecting block 2121 that forms the molding chamber, or a temperature monitoring element 2125 and a humidity monitoring element 2126 are disposed on a side of the second connecting block 2124 that forms the molding chamber; the temperature monitoring element 2125 disposed on the side of the first connecting block 2121 or on the side of the second connecting block 2124 is configured to monitor the temperature of the soil mass sample 40, and the humidity monitoring element 2126 disposed on the side of the first connecting block 2121 or on the side of the second connecting block 2124 is configured to monitor the humidity of the soil mass sample 40.

In this embodiment, by installing a temperature monitoring element 2125 and a humidity monitoring element 2126 on either the first connecting block 2121 or the second connecting block 2124 (or both), it is possible to monitor in real-time whether the temperature and humidity of the soil mass sample 40 are consistent with the environmental temperature and humidity inside the testing chamber 31, and whether the temperature and humidity state of the soil mass sample 40 has remained stable. When the state of the soil mass sample 40 is stable, coupling tests can be carried out, making the operation more convenient.

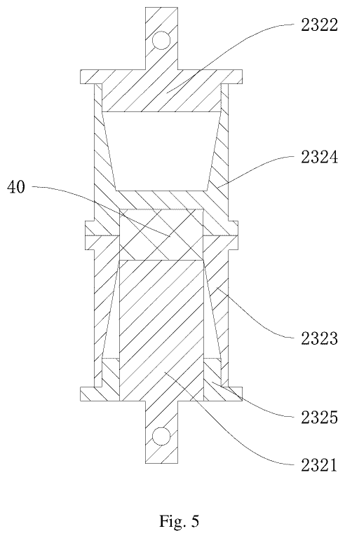

Referring to FIGS. 3 and 5, in some possible embodiments, the structure and working principle of the second shear testing assembly 23 are similar to those of the first shear testing assembly 21. The second shear testing assembly 23 includes a second slide rail 231, a second soil mass specimen former 232, a second shear loading unit 233, and a second shear top block 234. The second shear loading unit 233 and the second shear top block 234 are arranged opposite to each other relative to the soil mass sample 40. The second soil mass specimen former 232 is configured to prepare soil mass sample 40. The second soil mass specimen former 232 includes a third connecting block 2321, a fourth connecting block 2322, a third confining cylinder 2323, a fourth confining cylinder 2324, and a fifth confining cylinder 2325, which together form a cylindrical molding chamber for preparing a soil mass sample 40. The tension-compression loading assembly 24 is connected to either the third connecting block 2321 or the fourth connecting block 2322. That is, the second soil mass specimen former 232 can be installed upside-down, and subjected to a certain pressure to perform a compression-shear test. Similar to the first shear testing assembly 21, the second shear testing assembly 23 can also perform tension tests and shear tests independently.

In some possible embodiments, the first connecting block 2121 and the first constraining cylinder 2122 are connected together by a first snap-fit structure, and the second connecting block 2124 and the second constraining cylinder 2123 are connected together by a second snap-fit structure. The first snap-fit structure and the second snap-fit structure can be identical or different. This arrangement simplifies the locking mode compared with the thread locking mode and reduces the occurrence of soil jamming in the bolt threads.

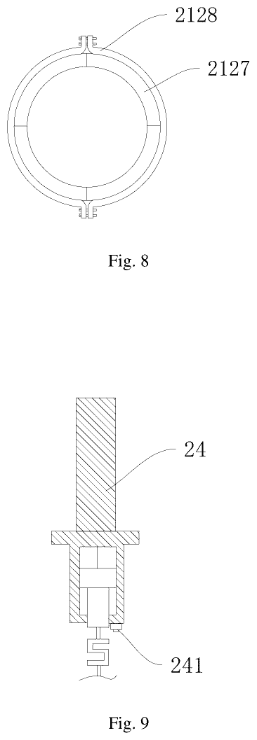

As shown in FIG. 8, in some possible embodiments, the first constraining cylinder 2122 and the second constraining cylinder 2123 are respectively formed by a plurality of arc-shaped plates 2127 enclosed and fixed together by at least one hoop ring 2128. The traditional fixing method adopts bolts for radial fastening, which tends to damage the soil mass during demolding. The fixing method using the hoop ring 2128 can reduce the number of bolts used, facilitate assembly and disassembly, avoid stress deviation caused by bolt tightening, and reduce disturbance to the internal soil mass.

As shown in FIG. 9, in some possible embodiments, the tension-compression loading assembly 24 is provided with a laser level 241, and the laser direction of the laser level 241 is vertical. Since tension tests, tension-shear tests and compression-shear tests all involve vertical force application, it is necessary to maintain the vertical state of the soil mass sample 40. The laser level 241 is provided to detect the verticality of the soil mass sample 40.

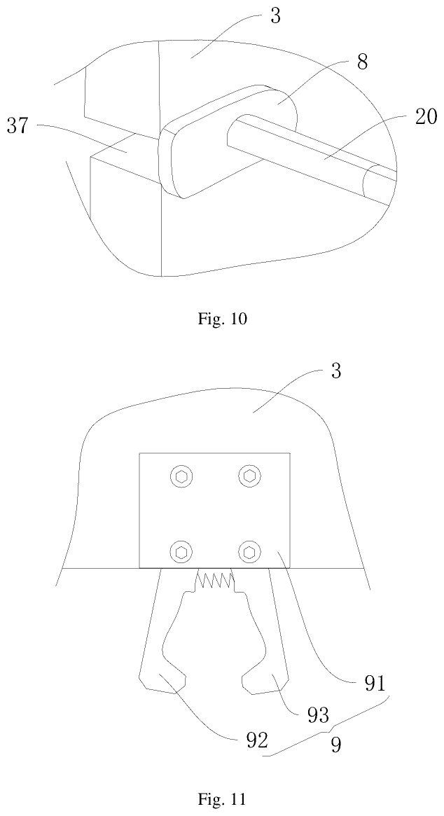

As shown in FIG. 1 and FIG. 10, in some possible embodiments, the box body 3 is provided with an avoidance opening 37 for avoiding the loading assembly 20, and the box body 3 is further provided with a blocking element for opening or closing the avoidance opening 37. According to the position of the loading assembly 20, different avoidance openings 37 can be arranged on the top wall, side wall and bottom wall of the box body 3. When no test is performed, the blocking member can be used to seal the avoidance opening 37 to ensure the cleanliness of the testing chamber 31. When a test is required, the blocking member is then removed.

As shown in FIG. 10, in some possible embodiments, the soil mass coupling test apparatus further includes a sealing ring 8 detachably arranged. When the loading assembly 20 is extended into the testing chamber 31 through the avoidance opening 37, the sealing ring 8 is arranged around the loading assembly 20 from the outside of the box body 3 to seal the avoidance opening 37. The arrangement of the sealing ring 8 can ensure that the temperature and humidity inside the testing chamber 31 are free from external interference, and realize air flow in accordance with the preset air outlet of the testing chamber 31.

As shown in FIG. 11, in some possible embodiments, the soil mass coupling test apparatus further includes a stopper 9 configured for locking the box body 3 at any position of the linear rail 2. The arrangement of the stopper 9 can ensure that the box body 3 is fixed at the test position, avoiding test errors caused by the shaking of the box body 3.

Specifically, the stopper 9 includes a mounting base 91 arranged on the outer wall of the box body 3, as well as a first claw 92 and a second claw 93 rotatably arranged on the mounting base 91. The first claw 92 and the second claw 93 can form a clamping space for clamping the linear rail 2. The linear rail 2 can be clamped by the first claw 92 and the second claw 93, so as to keep the box body 3 at any position. Of course, in other embodiments, the stopper 9 can also be a rotatable clamping block. A plurality of clamping holes are arranged on the linear rail 2, and the clamping effect is realized through the cooperation between the clamping block and the clamping holes, which is not specifically limited herein.

In the second aspect, the embodiment of the present application provides a test method for researching full-scale strength of soil mass adopting the apparatus provided in any of the above embodiments, which includes the following steps: determining the temperature and the humidity required for testing; fixing prepared soil mass samples 40 on the loading assembly 20, and moving the box body 3 from the idle position to the testing position, to accommodate the prepared soil mass samples 40 in the testing chamber 31 of the box body 3; closing the box door 4 of the box body 3, setting temperature parameters of the temperature control module 51 and humidity parameters of the humidity control module 52 based on a temperature and a humidity required for testing; waiting until a temperature and a humidity of the prepared soil mass samples 40 tend to be stable, controlling the loading assembly 20 to perform the soil mass coupling test on the prepared soil mass samples 40, and saving images and data of a test result; and after the soil mass coupling test is completed, adjusting the temperature control module 51 to a cooling state to freeze the prepared soil mass sample 40 that has been tested, and preserving a failure state of the prepared soil mass sample 40.

In some possible embodiments, a plurality of prepared soil mass samples 40 are provided; the loading assembly 20 is configured to perform a tension test, a tension-shear test, and a compression-shear test on the plurality of prepared soil mass samples 40 respectively, and after soil mass coupling tests on the plurality of prepared soil mass samples 40 are completed, a strength envelope of the plurality of prepared soil mass samples 40 is plotted based on the test results.

The test method for researching full-scale strength of soil mass provided in the embodiment of the present application can complete tension-shear tests, tension tests, and compression-shear tests on soil mass samples 40. During uniaxial tension and tension-shear tests, the shape of the soil mass samples 40 is designed to be thick at both ends and thin in the middle. The overall height of the soil mass sample 40 is 120 mm, and the middle section of the soil mass sample 40 is cylindrical, with a height of 20 mm and a diameter of 39 mm. The soil mass samples 40 for compression-shear tests are cylindrical with a height of 20 mm and a diameter of 39 mm, which not only ensures the application of tension force to soil mass samples 40 during uniaxial tension and tension-shear tests, and better transfers normal stress to the shear plane during compression-shear tests, but also ensures the uniformity of the size and shape of soil mass samples 40 near the failure surface, thereby accurately measuring the complete strength envelope of soil mass.

The loading assembly 20 can be driven by a servo motor, offering high loading accuracy. It supports various test force application modes, including constant stress, constant displacement, constant deformation, and load holding. Additionally, it can be programmed with specific cyclic steps as required, enabling shock-free switching among multiple control modes, thus achieving fully automatic closed-loop control.

The loading assembly 20 is controlled by a controller, which can be equipped with built-in measurement-and-control software. This software enables real-time measurement and display of various signals such as test force, peak value, displacement, and deformation, presenting live on-screen curves such as stress-strain and stress-displacement.

It is understandable that various parts in the above embodiments can be freely combined or deleted to form different combined embodiments. The specific contents of each combined embodiment will not be elaborated here. After this explanation, it can be considered that the specification of this application has already documented each combined embodiment and can support different combined embodiments.

The above description is merely preferred embodiments of the present application and is not intended to limit the scope of the present application. Any modifications, equivalent substitutions, and improvements made within the spirit and principles of the present application shall be included within the scope of protection of the present application.

What is claimed is:

1. A soil mass coupling test apparatus, comprising:
a soil mass testing machine, comprising a testing platform and a loading assembly arranged on the testing platform;
a linear rail, arranged horizontally at one side of the soil mass testing machine;
a box body, slidably arranged on the linear rail, wherein the box body comprises a testing chamber which has an inlet-outlet on a side facing towards the soil mass testing machine, the inlet-outlet being movably connected with a box door of the box body; and wherein the box body has a testing position and an idle position, and the loading assembly is at least partially accommodated within the testing chamber when the box body is in the testing position, and the loading assembly is disengaged from the testing chamber when the box body is in the idle position; and
an environment simulation assembly, comprising a temperature control module and a humidity control module, both of which are disposed in the box body, wherein the temperature control module is configured to adjust a temperature of the testing chamber, and the humidity control module is configured to adjust a humidity of the testing chamber.

2. The apparatus according to claim 1, wherein a wall of the testing chamber is provided with a heat exchange hole connected with an air duct, an output end of the temperature control module and an output end of the humidity control module being respectively connected with the air duct.

3. The apparatus according to claim 2, wherein the box body further comprises an equipment accommodating chamber; the temperature control module and the humidity control module are arranged in the equipment accommodating chamber; and the equipment accommodating chamber is in communication with the testing chamber through the air duct and the heat exchange hole.

4. The apparatus according to claim 2, further comprising:
a multi-blade centrifugal fan and a frequency converter arranged in the air duct to realize a dynamic regulation of an air volume; and
a flow deflector arranged in the air duct or the testing chamber;
wherein a cross-sectional shape of the air duct is circular, the air duct is configured to supply air from a top of the testing chamber and return air from a bottom of the testing chamber to form an vertical air flow, and the temperature control module and the humidity control module are respectively in communication with the air duct through a temperature channel and a humidity channel.

5. The apparatus according to claim 1, wherein the environmental simulation assembly further comprises an instrument detection module; and the instrument detection module is connected with the temperature control module and the humidity control module, and is configured to detect components in the temperature control module and the humidity control module.

6. The apparatus according to claim 1, wherein the soil mass testing machine further comprises a reaction frame, and the reaction frame is arranged on the testing platform and configured to form a testing area together with an upper surface of the testing platform, the testing area comprising a tension-shear zone, a tension zone and a compression-shear zone; the loading assembly comprises a first shear testing assembly arranged in the tension-shear zone, a tension testing assembly arranged in the tension zone, and a second shear testing assembly arranged in the compression-shear zone.

7. The apparatus according to claim 6, wherein the reaction frame comprises:
a top plate, disposed above the testing platform; and
two side plates, arranged opposite to each other and connected between the top plate and the testing platform, wherein the top plate, the two side plates and the upper surface of the testing platform enclose to form the testing area, the tension-shear zone being arranged adjacent to a first side plate of the two side plates, and the compression-shear zone being arranged adjacent to a second side plate of the two side plates.

8. The apparatus according to claim 6, wherein the first shear testing assembly comprises:
a first slide rail, arranged horizontally on the testing platform and located in the tension-shear zone;
a first soil mass specimen former, slidably arranged on the first slide rail;
a first shear loading unit, arranged on the reaction frame and having a first extensible end extendable and retractable along a length direction of the first slide rail, the first extensible end being provided with a first shear block; and
a first shear top block, arranged on the testing platform, wherein the first shear top block and the first shear block are arranged opposite to each other on two sides of the first soil mass specimen former and staggered in an vertical direction.

9. The apparatus according to claim 8, wherein the first shear testing assembly, the tension testing assembly, and the second shear testing assembly are arranged linearly, and the reaction frame has an adjustment slot above the first shear testing assembly, the tension testing assembly and the second shear testing assembly; and
the loading assembly further comprises a tension-compression loading assembly slidably arranged in the adjustment slot, the tension-compression loading assembly having a lifting end vertically movable.

10. The apparatus according to claim 9, wherein the first soil mass specimen former comprises:
a first connecting block, slidably fitted to the first slide rail;
a first confining cylinder, disposed above the first connecting block and detachably connected with the first connecting block;
a second confining cylinder, disposed above the first confining cylinder and detachably connected with the first confining cylinder; and
a second connecting block, disposed above the second confining cylinder and detachably connected with the second confining cylinder, an upper part of the second connecting block being detachably connected with the lifting end, wherein the first connecting block, the first confining cylinder, the second confining cylinder and the second connecting block form a molding chamber for a soil mass sample, a radial cross-section of the molding chamber being circular, and a diameter of the radial cross-section of the molding chamber gradually decreasing from two ends towards a middle of the molding chamber.

11. The apparatus according to claim 10, further comprising:
a temperature monitoring element and a humidity monitoring element disposed on a side of the first connecting block, or a temperature monitoring element and a humidity monitoring element disposed on a side of the second connecting block,
wherein the temperature monitoring element is configured to monitor a temperature of the soil mass sample, and the humidity monitoring element is configured to monitor a humidity of the soil mass sample.

12. The apparatus according to claim 10, wherein the first connecting block and the first constraining cylinder are connected together by a first snap-fit structure, and the second connecting block and the second constraining cylinder are connected together by a second snap-fit structure.

13. The apparatus according to claim 10, wherein the first constraining cylinder and the second constraining cylinder are respectively formed by a plurality of arc-shaped plates enclosed and fixed together by at least one hoop ring.

14. The apparatus according to claim 9, wherein the tension-compression loading assembly is provided with a laser level, and a laser direction of the laser level is vertical.

15. The apparatus according to claim 1, wherein the box body is provided with an avoidance opening configured for avoiding the loading assembly, and the box body is further provided with a blocking element configured for opening or closing the avoidance opening.

16. The apparatus according to claim 15, further comprising a sealing ring detachably arranged, wherein when the loading assembly is extended into the testing chamber through the avoidance opening, the sealing ring is arranged around the loading assembly from an outside of the box body to seal the avoidance opening.

17. The apparatus according to claim 1, further comprising a stopper configured for locking the box body at a position of the linear rail.

18. The apparatus according to claim 17, wherein the stopper comprises:
a mounting base arranged on an outer wall of the box body, and
a first claw and a second claw rotatably arranged on the mounting base and configured for forming a clamping space for clamping the linear rail.

19. A method for researching full-scale strength of soil mass, which adopts the apparatus according to claim 1, comprising:
determining a temperature and a humidity required for testing;
fixing a prepared soil mass sample on the loading assembly, and moving the box body from the idle position to the testing position, to accommodate the prepared soil mass sample in the testing chamber of the box body;
closing the box door of the box body, setting temperature parameters of the temperature control module and humidity parameters of the humidity control module based on a temperature and a humidity required for testing;
waiting until a temperature and a humidity of the prepared soil mass sample tend to be stable, controlling the loading assembly to perform a soil mass coupling test on the prepared soil mass sample, and saving images and data of a test result; and
after the soil mass coupling test is completed, adjusting the temperature control module to a cooling state to freeze the prepared soil mass sample that has been tested, and preserving a failure state of the prepared soil mass sample.

20. The method according to claim 19, wherein a plurality of prepared soil mass samples are provided, the loading assembly is configured to perform a tension test, a tension-shear test, and a compression-shear test on the plurality of prepared soil mass samples respectively, and after soil mass coupling tests on the plurality of prepared soil mass samples are completed, a strength envelope of the plurality of prepared soil mass samples is plotted based on test results.
